(19) United States
(12) Patent Application Publication
(10) Pub. No.: US 2005/0153946 A1
(43) Pub. Date: Jul. 14, 2005

(54) Temperature-stable formulations, and methods of development thereof

(75) Inventors: Jane Hirsh, Wellesley, MA (US); Donald Tibbetts, N. Falmouth, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Collegium Pharmaceuticals, Inc., Cumberland, RI (US)

(21) Appl. No.: 11/014,636

(22) Filed: Dec. 16, 2004

Related U.S. Application Data
(60) Provisional application No. 60/532,377, filed on Dec. 24, 2003.
(60) Provisional application No. 60/566,115, filed on Apr. 28, 2004.

(51) Int. Cl. A61K 31/56

(52) U.S. Cl. 514/170

(57) ABSTRACT

One embodiment of the present invention relates to a method of preparing a concentrated pharmaceutical formulation, comprising the steps of: combining in a container a therapeutic agent, a solvent and at least one pharmaceutically acceptable excipient to give a solution; adding to said solution a seed crystal of said compound to give a heterogeneous mixture; and observing the stability of said heterogeneous mixture.

### RELATED APPLICATIONS

This application claims the benefit of priority to U.S. Provisional Patent Application Ser. No. 60/532,377, filed Dec. 24, 2003; and U.S. Provisional Patent Application Ser. No. 60/566,115, filed Apr. 28, 2004; the specifications of which are hereby incorporated in their entirety.

### BACKGROUND OF THE INVENTION

An estimated forty-million Americans suffer from some form of rhinitis, sinusitis or a combination of both, e.g., rhinosinusitus. Allergic rhinitis is an inflammatory condition of the mucus membranes lining the nasal passages, caused by an allergy to pollen of trees, grasses, or weeds, or airborne mold spores, household dust mites, animal dandruff, and other substances. The allergic reaction causes nasal symptoms, such as sneezing, runny nose, itching, and congestion. Seasonal allergic rhinitis is commonly known as “hay fever” and is caused by allergens which are present at specific times of the year. Perennial allergic rhinitis is caused by allergens which are present in the environment year-round. Sinusitus, or inflammation of the paranasal sinuses is caused by viral, bacterial or fungal infections or may be secondary to other disorders such as allergy. Martindale The Complete Drug Reference, 32^{nd} Edition, The Pharmaceutical Press, London, UK.

The majority of the medications commonly prescribed for the treatment of these conditions include corticosteroids, antibacterials and antifungal agents, many of which are hydrophobic in nature and poorly soluble in water. Textbook of Organic Medicinal and Pharmaceutical Chemistry 10^{th} Edition, Delgado, Remers, Lippincott-Raven, Philadelphia, Pa. Stable aqueous formulations of these drugs are required for nasal administration. Stability requires a minimum concentration of solvent so as to reduce irritation while guaranteeing stability for stated storage conditions.

Steroidal anti-inflammatory agents, known for the treatment of such forms of rhinitis, are commonly available as nasal sprays. Examples of manual metered-dose steroidal nasal sprays commercially available as suspensions include Flonase (Fluticasone propionate) and Beconase AQ (Beclomethasone dipropionate) both by GlaxoSmithKline, Nasonex (Mometasone furoate monohydrate) by Schering, Rhinocort Aqua (Budesonide) by Astra Zeneca, and Nasacort (triamcinolone acetonide) by Aventis. Examples of manual metered-dose nasal sprays commercially available as aqueous solutions include Nasarel by Ivax (Flunisolide Nasal Solution). Muro Pharmaceutical received approval for a New Drug Application in February 2000 for Muro TriNasal® Spray (Triamcinolone acetonide 0.05% Nasal Solution); unfortunately, the product was recalled due to ongoing stability issues.

Nasal suspensions are pharmaceutical composition where the active ingredient is in the form of solid particles (generally in the range of 20 microns) that are dispersed in the aqueous phase of the formulation and suspended with the appropriate thixotropic agent to impart a viscosity similar to a gel (400-800 cps). Suspensions, due to their high viscosity, must typically be shaken prior to use by a patient. As the composition is shaken and subjected to sheer, the viscosity declines and allows the preparation to be administered in the form of a mist to the nasal mucosa. As the suspension dries the drug and the matrix of the thixotropic agent remain as residue on the nasal mucosa. The thixotropic agent has a drying effect that results in an adverse effect, epistaxis. Epistaxis, commonly called a nose-bleed, is reported for the various suspensions ranging from 2.7% to 11% within the Physician Desk Reference 2004. Other studies suggest generally ranging from 6-10% and one study as high as 18% consistent with long-term use and winter conditions. The nasal suspensions generally consist of the active ingredient in an aqueous medium containing a combination of various thixotropic agents, which can include glycerin microcrystalline cellulose, carboxymethylcellulose, dextrose, and the like. All nasal suspensions require vigorous shaking prior to use to ensure uniform delivery of the drug per application. The rheological profiles of commercial nasal-spray suspensions (Beconase, Nasacort, Flixonase) were compared using shear and extensional techniques. All the nasal suspensions were sheer thinning and were also thixotropic to varying degrees. The absence of significant thixotropic recovery at short times (5 minutes) for all the sprays implies that thixotropy is not necessarily the controlling factor for prolonged residence of the spray in the nasal cavity. Eccleston, G. M.; Rheological Behavior of Nasal Sprays in Sheer and Extension; Drug Dev. Ind. Pharm., 2000, 26, 975-983.

For example, an aqueous pharmaceutical suspension for nasal administration has been disclosed, comprising a pharmaceutically effective amount of solid particles of a medicament that is effective in treating a bodily condition by virtue of its being present on the mucosal surfaces of the nasal cavity; and a suspending agent in an amount effective to maintain said particles dispersed uniformly in the composition (U.S. Pat. No. 6,375,984). The aforementioned composition may be used to treat particular forms of rhinitis. U.S. Pat. No. 6,491,897 discloses stable nebulized solutions of budesonide solubulized in high concentrations of ethanol which must evaporate azetropically prior to inhalation into the body.

Aqueous steroidal nasal solutions typically consist of the active ingredients dissolved in the aqueous medium without thixotropic agents; consequently, the viscosity is much lower (approximately 40-50 cps) than a suspension. Nasal steroidal solutions do not require shaking prior to actuation; and they are simpler to manufacture and hence less expensive to produce. Propylene glycol employed as a solvent for the steroid is also a moisturizer and results in less epistaxis (Muro Tri-nasal 1.8%). Given these issues one would anticipate that these solutions would be commonly employed as the composition of choice. Unfortunately, they have suffered from significant stability issues involving precipitation when stored inadvertently at temperatures below stated storage conditions as defined by U.S. FDA (i.e., 20-25 C).

Recently, the United States Pharmacopeia (USP) identified risk factors that are encountered when therapeutic products move through the distribution chain (Hollander, R. et al. “Drug Products Distribution Chain.” Pharmacopeial Forum, vol. 29, no. 3, May-June 2003). These findings were based on examination of the entire distribution pathway in the United States, from the manufacturer to the end user or patient. The study concluded that further research must be done to assess the impact of extreme temperatures and humidity on the efficacy of drugs.

### SUMMARY OF THE INVENTION

One embodiment of the present invention relates to a method of preparing a concentrated pharmaceutical formulation, comprising the steps of: combining in a container a therapeutic agent, a solvent and at least one pharmaceutically acceptable excipient to give a solution; adding to said solution a seed crystal of said compound to give a heterogeneous mixture; and observing the stability of said heterogeneous mixture.

In a preferred embodiment the instant invention provides a way of stabilizing hydrophobic drugs in water-containing formulae against precipitation on storage in the cold.

One embodiment of the present invention relates to an aqueous formulation, comprising water; a therapeutic agent, selected from the group consisting of anti-inflammatory steroids and steroidal hormones, in an amount between about 0.001% and about 2.0% (w/v); propylene glycol in an amount between about 13% and about 20% (w/v); polyethylene glycol (PEG) in an amount between about 10% and about 50% (w/v); a preservative; a stabilizer; and a pH buffering agent sufficient to maintain the pH of the aqueous formulation at between about 3.5 and about 8.0.

In certain embodiments, the present invention relates to the aforementioned formulations and the attendant definitions, wherein said formulation is stable at storage conditions at about 20° C. to about 25° C.

In certain embodiments, the present invention relates to the aforementioned formulations and the attendant definitions, wherein the amount of propylene glycol is about 14% (w/v).

In certain embodiments, the present invention relates to the aforementioned formulations and the attendant definitions, wherein the amount of preservative is between about 0.01% and about 0.08% (w/v).

In certain embodiments, the present invention relates to the aforementioned formulations and the attendant definitions, wherein the amount of stabilizer is between about 0.005% and about 0.05% (w/v).

In certain embodiments, the present invention relates to the aforementioned formulations and the attendant definitions, wherein the preservative is benzalkonium chloride.

In certain embodiments, the present invention relates to the aforementioned formulations and the attendant definitions, wherein the stabilizer is disodium ethylenediaminetetraacetic acid (EDTA).

In certain embodiments, the present invention relates to the aforementioned formulations and the attendant definitions, wherein the therapeutic agent is a steroidal hormone selected from the group consisting of estrogens, progestins, androgens, and mixtures of any of them.

In certain embodiments, the present invention relates to the aforementioned formulations and the attendant definitions, wherein the therapeutic agent is a steroidal hormone selected from the group consisting of benzestrol, broparoestriol, chlorotrianisene, clopormon, desogesterol, dienestriol, equilenin, equilin, estradiol, estriol, estrone, ethinyl estradiol, gestodene, hexestrol, lynestrenol, mestranol, methallenestril, methestrol, moxestriol, mytatrienediol, norethindrone, norethynodrel, norgestimate, quinestradiol, quinestrol, allylestrenol, altrenogest, anagestone, chlormadinone acetate, delmadinone acetate, demegestone, dimethisterone, drospirenone, dydrogesterone, ethisterone, ethynodiol, flurogestone acetate, gestonorone caproate, 17-hydroxy-16-methylene-X⁶-progesterone, 17α-hydroxyprogesterone, medrogestone, medroxyprogesterone, megestrol acetate, melengestrol, norgesterone, norgestrel, norgestrienone, norvinisterone, pentagesterone, progesterone, promegestrone, trengestrone, boldenone, cloxotestosterone, fluoxymesterone, mesterolone, methandrostenolone, 17-methyltestosterone, 17α-methyltestosterone-3-cylcopentyl enol ether, mibolerone, norethandrolone, normethandrone, oxandrolone, oxymesterone, oxymetholone, stanolone, stanozolol, testosterone, tismesterone, and mixtures of any of them.

In certain embodiments, the present invention relates to the aforementioned formulations and the attendant definitions, wherein the therapeutic agent is an anti-inflammatory steroid selected from the group consisting of 21-acetoxypregnenolone, alclometasone, algestone, alisactide, amcinonide, aminoglutethimide, beclomethasone, beclomethasone dipropionate, betamethasone, betamethasone dipropionate, betamethasone adamantoate, budesonide, butixocort, chloropredinisone, ciclometasone, clobetasol, clobetasone, clocortolone, cloprednol, corticosterone, cortisone, cortivazol, deflazacort, deprodone, deprodone propionate, desonide, desoximetasone, dexamethasone, dexamethasonisonicotinate, diflorasone, diflucortolone, difluprednate, endrisone, enoxolone, fluazacort, flucloronide, flumethasone, flunisolide, fluocinolone acetonide, fluocinonide, flucortin, fluocortin butyl, flodexan fluclorolone acetonide, fluocortolone, fluorometholone, fluperolone acetate, fluprednidene acetate, fluprednisolone, flurandrenolide, fluticasone propionate, formebolone, formocortal, halcinonide, halobetasol propionate, halometasone, halopredone acetate hydrocortamate, hydrocortisone, hydrocortisone aceponate, hydrocortisone butyrate, hydrocortisone-17-butyrate, icomethasone enbutyate, loteprednol etabonate, lotrisone, mazipredone, medrysone, meprednisone, methylprednisolone, mometasone furoate, mometasone furoate monohydrate, mycophenolate mofetil, paramethasone, pranlukast, prednicarbate, prednisolone, prednisolone 25-diethylaminoacetate, prednisolone sodium phosphate, prednisone, prednival, prednylidene promedrol, rimexolone, seratrodast, tipredane, tixocortol, triamcinolone, triamcinolone acetonide, triamcinolone benetonide, triamcinolone hexacetonide, triiostane, ulobetasol propionate, zileuton, and mixtures of any of them.

In certain embodiments, the present invention relates to the aforementioned formulations and the attendant definitions, wherein the therapeutic agent is triamcinolone acetonide.

In certain embodiments, the present invention relates to the aforementioned formulations and the attendant definitions, wherein the amount of propylene glycol is about 14% (w/v).

In certain embodiments, the present invention relates to the aforementioned formulations and the attendant definitions, wherein the amount of preservative is between about 0.01% and about 0.08% (w/v).

In certain embodiments, the present invention relates to the aforementioned formulations and the attendant definitions, wherein the amount of stabilizer is between about 0.005% and about 0.05% (w/v).

In certain embodiments, the present invention relates to the aforementioned formulations and the attendant definitions, wherein the preservative is benzalkonium chloride.

In certain embodiments, the present invention relates to the aforementioned formulations and the attendant definitions, wherein the stabilizer is disodium ethylenediaminetetraacetic acid (EDTA).

One embodiment of the present invention relates to an aqueous formulation, comprising water; triamcinolone acetonide in an amount between about 0.01% and about 0.05% (w/v); propylene glycol in an amount of about 14% (w/v); PEG in an amount between about 35% and 45% (w/v); benzalkonium chloride in an amount of about 0.05% (w/v); disodium EDTA in an amount of about 0.05% (w/v); citric acid in an amount of about 0.72% (w/v); sodium citrate dihydrate in an amount of about 0.74% (w/v); and an amount of a pH buffering agent sufficient to maintain the pH of the aqueous formulation between about 5 and 7.

One embodiment of the present invention relates to an aqueous formulation, comprising a solution comprising an anti-inflammatory steroid, a thickening agent, an organic solvent, and water; a metal or plastic or glass bottle comprising a concave or convex interior bottom, a dip tube, and a cap comprising a metered-dose manual spray pump that when activated emits a mist; wherein the aqueous formulation has a viscosity between about 45 cps and about 50 cps, and a specific gravity at about 25° C. of about 1.070 to about 1.090.

In certain embodiments, the present invention relates to the aforementioned formulations and the attendant definitions, wherein the formulation is stable at storage conditions at about 20° C. to about 25° C.

In certain embodiments, the present invention relates to the aforementioned formulations and the attendant definitions, wherein the anti-inflammatory steroid is selected from the group consisting of 21-acetoxypregnenolone, alclometasone, algestone, alisactide, amcinonide, aminoglutethimide, beclomethasone, beclomethasone dipropionate, betamethasone, betamethasone dipropionate, betamethasone adamantoate, budesonide, butixocort, chloropredinisone, ciclometasone, clobetasol, clobetasone, clocortolone, cloprednol, corticosterone, cortisone, cortivazol, deflazacort, deprodone, deprodone propionate, desonide, desoximetasone, dexamethasone, dexamethasonisonicotinate, diflorasone, diflucortolone, difluprednate, endrisone, enoxolone, fluazacort, flucloronide, flumethasone, flunisolide, fluocinolone acetonide, fluocinonide, flucortin, fluocortin butyl, flodexan fluclorolone acetonide, fluocortolone, fluorometholone, fluperolone acetate, fluprednidene acetate, fluprednisolone, flurandrenolide, fluticasone propionate, formebolone, formocortal, halcinonide, halobetasol propionate, halometasone, halopredone acetate hydrocortamate, hydrocortisone, hydrocortisone aceponate, hydrocortisone butyrate, hydrocortisone-17-butyrate, icomethasone enbutyate, loteprednol etabonate, lotrisone, mazipredone, medrysone, meprednisone, methylprednisolone, mometasone furoate, mometasone furoate monohydrate, mycophenolate mofetil, paramethasone, pranlukast, prednicarbate, prednisolone, prednisolone 25-diethylaminoacetate, prednisolone sodium phosphate, prednisone, prednival, prednylidene promedrol, rimexolone, seratrodast, tipredane, tixocortol, triamcinolone, triamcinolone acetonide, triamcinolone benetonide, triamcinolone hexacetonide, triiostane, ulobetasol propionate, zileuton, and mixtures of any of them.

In certain embodiments, the present invention relates to the aforementioned formulations and the attendant definitions, wherein the organic solvent is propylene glycol.

In certain embodiments, the present invention relates to the aforementioned formulations and the attendant definitions, wherein the thickening agent is PEG.

In certain embodiments, the present invention relates to the aforementioned formulations and the attendant definitions, wherein the anti-inflammatory steroid is selected from the group consisting of 21-acetoxypregnenolone, alclometasone, algestone, alisactide, amcinonide, aminoglutethimide, beclomethasone, beclomethasone dipropionate, betamethasone, betamethasone dipropionate, betamethasone adamantoate, budesonide, butixocort, chloropredinisone, ciclometasone, clobetasol, clobetasone, clocortolone, cloprednol, corticosterone, cortisone, cortivazol, deflazacort, deprodone, deprodone propionate, desonide, desoximetasone, dexamethasone, dexamethasonisonicotinate, diflorasone, diflucortolone, difluprednate, endrisone, enoxolone, fluazacort, flucloronide, flumethasone, flunisolide, fluocinolone acetonide, fluocinonide, flucortin, fluocortin butyl, flodexan fluclorolone acetonide, fluocortolone, fluorometholone, fluperolone acetate, fluprednidene acetate, fluprednisolone, flurandrenolide, fluticasone propionate, formebolone, formocortal, halcinonide, halobetasol propionate, halometasone, halopredone acetate hydrocortamate, hydrocortisone, hydrocortisone aceponate, hydrocortisone butyrate, hydrocortisone-17-butyrate, icomethasone enbutyate, loteprednol etabonate, lotrisone, mazipredone, medrysone, meprednisone, methylprednisolone, mometasone furoate, mometasone furoate monohydrate, mycophenolate mofetil, paramethasone, pranlukast, prednicarbate, prednisolone, prednisolone 25-diethylaminoacetate, prednisolone sodium phosphate, prednisone, prednival, prednylidene promedrol, rimexolone, seratrodast, tipredane, tixocortol, triamcinolone, triamcinolone acetonide, triamcinolone benetonide, triamcinolone hexacetonide, triiostane, ulobetasol propionate, zileuton, and mixtures of any of them; the organic solvent is propylene glycol; and the thickening agent is PEG.

In certain embodiments, the present invention relates to the aforementioned formulations and the attendant definitions, wherein the anti-inflammatory steroid is triamcinolone acetonide; the organic solvent is propylene glycol; and the thickening agent is PEG.

In certain embodiments, the present invention relates to the aforementioned formulations and the attendant definitions, wherein the anti-inflammatory steroid is triamcinolone acetonide at a concentration of about 0.05% (w/v); the thickening agent is PEG at a concentration of about 40%; and the organic solvent is propylene glycol at a concentration of about 14%.

One embodiment of the present invention relates to a kit comprising any one of the aforementioned aqueous formulations.

In certain embodiments, the present invention relates to the aforementioned kits, wherein said aqueous formulation further comprises an antihistamine, decongestant, ophthalmological, antibiotic, antifungal or irrigating solution.

In certain embodiments, the present invention relates to the aforementioned kits, further comprising a solid or liquid dosage form of an antihistamine, decongestant, mucolytic agent, ophthalmological, or antibiotic.

In certain embodiments, the present invention relates to the aforementioned kits, further comprising a separate irrigating solution.

One embodiment of the present invention relates to a method of treating inflammation of a nasal mucosa or paranasal mucosa in a subject, comprising intranasally administering to a subject in need thereof a therapeutically effective amount of an aqueous formulation of any of claims 1 to 7 or 10 to 25.

In certain embodiments, the present invention relates to any of the aforementioned methods and the attendant definitions, wherein the therapeutically effective amount of the therapeutic agent is about 25 micrograms to about 600 micrograms per day.

One embodiment of the present invention relates to a method for developing a temperature-stable formulation of a therapeutic agent, comprising the steps of preparing in a plurality of containers a plurality of formulations, wherein each formulation comprises an amount of a first solvent, an amount of a second solvent, an amount of a therapeutic agent in solution, and a solid sample of the therapeutic agent; wherein said amount of said second solvent is not the same in all of the containers; subjecting the plurality of containers to one or more temperatures for one or more periods of time; determining for each container the concentration of said therapeutic agent in solution or whether a solid sample of the therapeutic agent is present or the quantity of the solid sample of the therapeutic agent or any of them; and selecting one or more containers wherein no solid sample of the therapeutic agent is present or the quantity of said solid sample of said therapeutic agent has not increased.

In certain embodiments, the present invention relates to any of the aforementioned methods and the attendant definitions, wherein said solid sample of the therapeutic agent adheres to the container walls.

In certain embodiments, the present invention relates to any of the aforementioned methods and the attendant definitions, wherein said solid sample of the therapeutic agent is suspended in the solutions.

In certain embodiments, the present invention relates to any of the aforementioned methods and the attendant definitions, wherein said containers are the same or similar to the containers that will store the temperature-stable formulation over a long term period.

In certain embodiments, the present invention relates to any of the aforementioned methods and the attendant definitions, wherein said one or more temperatures are selected from the range of temperatures from about 0° C. to about 40° C.

In certain embodiments, the present invention relates to any of the aforementioned methods and the attendant definitions, wherein said first solvent is water and said second solvent is an organic solvent.

In certain embodiments, the present invention relates to any of the aforementioned methods and the attendant definitions, wherein the temperature-stable formulations comprise from about 2% to about 70% (w/v) of said second solvent, wherein said second solvent is an organic solvent.

In certain embodiments, the present invention relates to any of the aforementioned methods and the attendant definitions, wherein said therapeutic agent is active when administered by a route selected from a nasal spray, an inhalation delivery device, eye drops, ear drops, or nose drops.

In certain embodiments, the present invention relates to any of the aforementioned methods and the attendant definitions, wherein said therapeutic agent is a steroid, an antifungal, an antibiotic or an antimicrobial.

In certain embodiments, the present invention relates to any of the aforementioned methods and the attendant definitions, wherein said therapeutic agent is a steroid selected from the group consisting of estrogens, progestins, androgens, and mixtures of any of them.

In certain embodiments, the present invention relates to any of the aforementioned methods and the attendant definitions, wherein said therapeutic agent is selected from the group consisting of benzestrol, broparoestriol, chlorotrianisene, clopormon, desogesterol, dienestriol, equilenin, equilin, estradiol, estriol, estrone, ethinyl estradiol, gestodene, hexestrol, lynestrenol, mestranol, methallenestril, methestrol, moxestriol, mytatrienediol, norethindrone, norethynodrel, norgestimate, quinestradiol, quinestrol, allylestrenol, altrenogest, anagestone, chlormadinone acetate, delmadinone acetate, demegestone, dimethisterone, drospirenone, dydrogesterone, ethisterone, ethynodiol, flurogestone acetate, gestonorone caproate, 17-hydroxy-16-methylene-X⁶-progesterone, 17α-hydroxyprogesterone, medrogestone, medroxyprogesterone, megestrol acetate, melengestrol, norgesterone, norgestrel, norgestrienone, norvinisterone, pentagesterone, progesterone, promegestrone, trengestrone, boldenone, cloxotestosterone, fluoxymesterone, mesterolone, methandrostenolone, 17-methyltestosterone, 17α-methyltestosterone-3-cylcopentyl enol ether, mibolerone, norethandrolone, normethandrone, oxandrolone, oxymesterone, oxymetholone, stanolone, stanozolol, testosterone, tismesterone, and mixtures of any of them.

In certain embodiments, the present invention relates to any of the aforementioned methods and the attendant definitions, wherein said therapeutic agent is an anti-inflammatory steroid.

In certain embodiments, the present invention relates to any of the aforementioned methods and the attendant definitions, wherein said therapeutic agent is an anti-inflammatory steroid selected from the group consisting of 21-acetoxypregnenolone, alclometasone, algestone, alisactide, amcinonide, aminoglutethimide, beclomethasone, beclomethasone dipropionate, betamethasone, betamethasone dipropionate, betamethasone adamantoate, budesonide, butixocort, chloropredinisone, ciclometasone, clobetasol, clobetasone, clocortolone, cloprednol, corticosterone, cortisone, cortivazol, deflazacort, deprodone, deprodone propionate, desonide, desoximetasone, dexamethasone, dexamethasonisonicotinate, diflorasone, diflucortolone, difluprednate, endrisone, enoxolone, fluazacort, flucloronide, flumethasone, flunisolide, fluocinolone acetonide, fluocinonide, flucortin, fluocortin butyl, flodexan fluclorolone acetonide, fluocortolone, fluorometholone, fluperolone acetate, fluprednidene acetate, fluprednisolone, flurandrenolide, fluticasone propionate, formebolone, formocortal, halcinonide, halobetasol propionate, halometasone, halopredone acetate hydrocortamate, hydrocortisone, hydrocortisone aceponate, hydrocortisone butyrate, hydrocortisone-17-butyrate, icomethasone enbutyate, loteprednol etabonate, lotrisone, mazipredone, medrysone, meprednisone, methylprednisolone, mometasone furoate, mometasone furoate monohydrate, mycophenolate mofetil, paramethasone, pranlukast, prednicarbate, prednisolone, prednisolone 25-diethylaminoacetate, prednisolone sodium phosphate, prednisone, prednival, prednylidene promedrol, rimexolone, seratrodast, tipredane, tixocortol, triamcinolone, triamcinolone acetonide, triamcinolone benetonide, triamcinolone hexacetonide, triiostane, ulobetasol propionate, zileuton, and mixtures of any of them.

In certain embodiments, the present invention relates to any of the aforementioned methods and the attendant definitions, wherein said therapeutic agent is triamcinolone acetonide.

In certain embodiments, the present invention relates to any of the aforementioned methods and the attendant definitions, wherein said second solvent is a water-miscible biocompatible organic solvent or mixture of them.

In certain embodiments, the present invention relates to any of the aforementioned methods and the attendant definitions, wherein the solid sample of said therapeutic agent is obtained by preparing a saturated or supersaturated solution of said therapeutic agent at a first temperature and storing the supersaturated solution at a second temperature, wherein said first temperature is higher than said second temperature.

In certain embodiments, the present invention relates to any of the aforementioned methods and the attendant definitions, wherein said first solvent is water; wherein said temperature-stable formulation is suitable for administration via a nasal spray, an inhalation delivery device, eye drops, ear drops, or nose drops.

In certain embodiments, the present invention relates to any of the aforementioned methods and the attendant definitions, wherein said therapeutic agent is an anti-inflammatory steroid; and said container is metal or plastic, further comprising a concave or convex interior bottom, a dip tube, and a cap comprising a metered-dose manual spray pump that when activated emits a mist.

In certain embodiments, the present invention relates to any of the aforementioned methods and the attendant definitions, wherein the anti-inflammatory steroid is selected from the group consisting of 21-acetoxypregnenolone, alclometasone, algestone, alisactide, amcinonide, aminoglutethimide, beclomethasone, beclomethasone dipropionate, betamethasone, betamethasone dipropionate, betamethasone adamantoate, budesonide, butixocort, chloropredinisone, ciclometasone, clobetasol, clobetasone, clocortolone, cloprednol, corticosterone, cortisone, cortivazol, deflazacort, deprodone, deprodone propionate, desonide, desoximetasone, dexamethasone, dexamethasonisonicotinate, diflorasone, diflucortolone, difluprednate, endrisone, enoxolone, fluazacort, flucloronide, flumethasone, flunisolide, fluocinolone acetonide, fluocinonide, flucortin, fluocortin butyl, flodexan fluclorolone acetonide, fluocortolone, fluorometholone, fluperolone acetate, fluprednidene acetate, fluprednisolone, flurandrenolide, fluticasone propionate, formebolone, formocortal, halcinonide, halobetasol propionate, halometasone, halopredone acetate hydrocortamate, hydrocortisone, hydrocortisone aceponate, hydrocortisone butyrate, hydrocortisone-17-butyrate, icomethasone enbutyate, loteprednol etabonate, lotrisone, mazipredone, medrysone, meprednisone, methylprednisolone, mometasone furoate, mometasone furoate monohydrate, mycophenolate mofetil, paramethasone, pranlukast, prednicarbate, prednisolone, prednisolone 25-diethylaminoacetate, prednisolone sodium phosphate, prednisone, prednival, prednylidene promedrol, rimexolone, seratrodast, tipredane, tixocortol, triamcinolone, triamcinolone acetonide, triamcinolone benetonide, triamcinolone hexacetonide, triiostane, ulobetasol propionate, zileuton, and mixtures of any of them.

In certain embodiments, the present invention relates to any of the aforementioned methods and the attendant definitions, wherein the anti-inflammatory steroid is triamcinolone acetonide.

In certain embodiments, the present invention relates to any of the aforementioned methods and the attendant definitions, wherein the temperature-stable formulation comprises one or more therapeutic agents selected from the group consisting of steroids, antifungals, antibiotics and antimicrobials.

In certain embodiments, the present invention relates to any of the aforementioned methods and the attendant definitions, wherein the temperature-stable formulations comprise from about 2% to about 70% (w/v) of said second solvent, wherein said second solvent is an organic solvent.

In certain embodiments, the present invention relates to any of the aforementioned methods and the attendant definitions, wherein the temperature-stable formulation further comprises a thickening agent.

In certain embodiments, the present invention relates to any of the aforementioned methods and the attendant definitions, wherein the temperature-stable formulation has a viscosity between about 30 cps and about 400 cps.

In certain embodiments, the present invention relates to any of the aforementioned methods and the attendant definitions, wherein said therapeutic agent is an anti-inflammatory steroid selected from the group consisting of 21-acetoxypregnenolone, alclometasone, algestone, alisactide, amcinonide, aminoglutethimide, beclomethasone, beclomethasone dipropionate, betamethasone, betamethasone dipropionate, betamethasone adamantoate, budesonide, butixocort, chloropredinisone, ciclometasone, clobetasol, clobetasone, clocortolone, cloprednol, corticosterone, cortisone, cortivazol, deflazacort, deprodone, deprodone propionate, desonide, desoximetasone, dexamethasone, dexamethasonisonicotinate, diflorasone, diflucortolone, difluprednate, endrisone, enoxolone, fluazacort, flucloronide, flumethasone, flunisolide, fluocinolone acetonide, fluocinonide, flucortin, fluocortin butyl, flodexan fluclorolone acetonide, fluocortolone, fluorometholone, fluperolone acetate, fluprednidene acetate, fluprednisolone, flurandrenolide, fluticasone propionate, formebolone, formocortal, halcinonide, halobetasol propionate, halometasone, halopredone acetate hydrocortamate, hydrocortisone, hydrocortisone aceponate, hydrocortisone butyrate, hydrocortisone-17-butyrate, icomethasone enbutyate, loteprednol etabonate, lotrisone, mazipredone, medrysone, meprednisone, methylprednisolone, mometasone furoate, mometasone furoate monohydrate, mycophenolate mofetil, paramethasone, pranlukast, prednicarbate, prednisolone, prednisolone 25-diethylaminoacetate, prednisolone sodium phosphate, prednisone, prednival, prednylidene promedrol, rimexolone, seratrodast, tipredane, tixocortol, triamcinolone, triamcinolone acetonide, triamcinolone benetonide, triamcinolone hexacetonide, triiostane, ulobetasol propionate, zileuton, and mixtures of any of them; and wherein the temperature-stable formulation further comprises from about 2% to about 70% (w/v) of said second solvent, wherein said second solvent is an organic solvent.

In certain embodiments, the present invention relates to any of the aforementioned methods and the attendant definitions, wherein said therapeutic agent is an anti-inflammatory steroid selected from the group consisting of 21-acetoxypregnenolone, alclometasone, algestone, alisactide, amcinonide, aminoglutethimide, beclomethasone, beclomethasone dipropionate, betamethasone, betamethasone dipropionate, betamethasone adamantoate, budesonide, butixocort, chloropredinisone, ciclometasone, clobetasol, clobetasone, clocortolone, cloprednol, corticosterone, cortisone, cortivazol, deflazacort, deprodone, deprodone propionate, desonide, desoximetasone, dexamethasone, dexamethasonisonicotinate, diflorasone, diflucortolone, difluprednate, endrisone, enoxolone, fluazacort, flucloronide, flumethasone, flunisolide, fluocinolone acetonide, fluocinonide, flucortin, fluocortin butyl, flodexan fluclorolone acetonide, fluocortolone, fluorometholone, fluperolone acetate, fluprednidene acetate, fluprednisolone, flurandrenolide, fluticasone propionate, formebolone, formocortal, halcinonide, halobetasol propionate, halometasone, halopredone acetate hydrocortamate, hydrocortisone, hydrocortisone aceponate, hydrocortisone butyrate, hydrocortisone-17-butyrate, icomethasone enbutyate, loteprednol etabonate, lotrisone, mazipredone, medrysone, meprednisone, methylprednisolone, mometasone furoate, mometasone furoate monohydrate, mycophenolate mofetil, paramethasone, pranlukast, prednicarbate, prednisolone, prednisolone 25-diethylaminoacetate, prednisolone sodium phosphate, prednisone, prednival, prednylidene promedrol, rimexolone, seratrodast, tipredane, tixocortol, triamcinolone, triamcinolone acetonide, triamcinolone benetonide, triamcinolone hexacetonide, triiostane, ulobetasol propionate, zileuton, and mixtures of any of them; the temperature-stable formulation further comprises from about 2% to about 70% (w/v) of said second solvent, wherein said second solvent is an organic solvent; and wherein the temperature-stable formulation further comprises a thickening agent.

In certain embodiments, the present invention relates to any of the aforementioned methods and the attendant definitions, wherein said therapeutic agent is an anti-inflammatory steroid selected from the group consisting of 21-acetoxypregnenolone, alclometasone, algestone, alisactide, amcinonide, aminoglutethimide, beclomethasone, beclomethasone dipropionate, betamethasone, betamethasone dipropionate, betamethasone adamantoate, budesonide, butixocort, chloropredinisone, ciclometasone, clobetasol, clobetasone, clocortolone, cloprednol, corticosterone, cortisone, cortivazol, deflazacort, deprodone, deprodone propionate, desonide, desoximetasone, dexamethasone, dexamethasonisonicotinate, diflorasone, diflucortolone, difluprednate, endrisone, enoxolone, fluazacort, flucloronide, flumethasone, flunisolide, fluocinolone acetonide, fluocinonide, flucortin, fluocortin butyl, flodexan fluclorolone acetonide, fluocortolone, fluorometholone, fluperolone acetate, fluprednidene acetate, fluprednisolone, flurandrenolide, fluticasone propionate, formebolone, formocortal, halcinonide, halobetasol propionate, halometasone, halopredone acetate hydrocortamate, hydrocortisone, hydrocortisone aceponate, hydrocortisone butyrate, hydrocortisone-17-butyrate, icomethasone enbutyate, loteprednol etabonate, lotrisone, mazipredone, medrysone, meprednisone, methylprednisolone, mometasone furoate, mometasone furoate monohydrate, mycophenolate mofetil, paramethasone, pranlukast, prednicarbate, prednisolone, prednisolone 25-diethylaminoacetate, prednisolone sodium phosphate, prednisone, prednival, prednylidene promedrol, rimexolone, seratrodast, tipredane, tixocortol, triamcinolone, triamcinolone acetonide, triamcinolone benetonide, triamcinolone hexacetonide, triiostane, ulobetasol propionate, zileuton, and mixtures of any of them; the temperature-stable formulation further comprises from about 2% to about 70% (w/v) of said second solvent, wherein said second solvent is an organic solvent; the temperature-stable formulation further comprises a thickening agent; and wherein the temperature-stable formulation has a viscosity between about 30 cps and about 400 cps.

In certain embodiments, the present invention relates to any of the aforementioned methods and the attendant definitions, wherein said one or more temperatures are selected from the range of temperatures from about 0° C. to about 40° C.; and said period of time is greater than or equal to eight weeks.

One embodiment of the present invention relates to an aqueous formulation, comprising water; a poorly-soluble therapeutic agent in an concentration between about 0.01% and about 0.2% (w/v), wherein said drug is not soluble in water to a critical therapeutic concentration; propylene glycol in an amount between about 2% to about 20% (w/v); polyethylene glycol (PEG) in an amount between about 10% and about 50% (w/v); a preservative; optionally a stabilizer; and a pH buffering agent sufficient to maintain the pH of the aqueous formulation at between about 3.5 and about 8.0.

In certain embodiments, the present invention relates to any of the aforementioned methods and the attendant definitions, wherein said poorly-soluble therapeutic agent is not soluble in water to a critical therapeutic concentration between about 0.01% and about 0.2% (w/v).

In certain embodiments, the present invention relates to any of the aforementioned methods and the attendant definitions, wherein said poorly soluble therapeutic agent is triamcinolone acetonide.

These and other embodiments of the present invention, and their features and characteristics, will be apparent from the description, drawings and claims that follow.

### BRIEF DESCRIPTION OF DRAWINGS

FIG. 1 depicts a plot of the solubility versus temperature for formulations comprising varying amounts of propylene glycol.

FIG. 2 depicts a plot of the solubility versus temperature for formulations comprising varying amounts of propylene glycol, wherein the results are presented in a graph where all assays above 100% have been truncated to 100%.

### DETAILED DESCRIPTION OF THE INVENTION

Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used to mean “including but not limited to”. “Including” and “including but not limited to” are used interchangeably.

The term “active agent” or “therapeutic agent” is art-recognized and refers to any chemical moiety that is a biologically, physiologically, or pharmacologically active substance that acts locally or systemically in a subject. Examples of active or therapeutic agents, also referred to as “drugs”, are described in well-known literature references such as the Merck Index, the Physicians Desk Reference, and The Pharmacological Basis of Therapeutics, and they include, without limitation, medicaments; steroids; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Anti-inflammatory steroids are examples of active agents.

The term “screening” is art recognized and refers to a system for preliminary appraisal and selection of a formulation based on its suitability for a particular use and conditions.

The terms “stable” and “stable formulation” are related and are used herein to refer to a formulation that maintains a relatively homogeneous distribution of active agent.

The phrase “stable formulation over a long term period” is used herein to refer to the reasonable time that a commercial product comprising the formulation of the present invention will take to go from manufacture to use. The phrase also takes into consideration the conditions that the commercial product will be exposed to, including, for example, temperature.

The term “optimal” as used herein refers to a percentage of a formulation component that gives an acceptable balance between formulation stability and undesirable side effects. For example, an optimal percentage of propylene glycol in the formulations of the present invention is one that results in a formulation stable over a long term period but has very little or no stinging, poor taste, or poor mouth feel side effects.

The term “seed crystals” is art recognized and refers to a small amount of material that serves as a nucleus for initiating a desired reaction. For example, a small crystal used to start the growth process of a large crystal.

The term “saturated” is art recognized and refers to a solution wherein the solution contains a sufficient amount of a substance so that no more will dissolve under the given conditions; e.g., the concentration of dissolved solute is or would be in equilibrium with any excess undissolved solute; the undissolved solute need not actually be present for the description to apply.

The term “supersaturated” is art recognized and refers to a solution wherein the solution holds more of a dissolved solute than is required to produce equilibrium with its undissolved solute.

The term “inflammation” is art recognized and refers to a protective response of tissues affected by disease or injury, and characterized by redness, localized heat, swelling, pain, and possibly impaired function of the affected part.

The term “anti-inflammatory” is art recognized and refers to an agent that counteracts or suppresses inflammation without acting directly against the cause.

The term “steroid” is art recognized and refers to any of a class of compounds including the sterols, bile acids, sex hormones, and adrenocortical hormones; all of which comprise the ring structure (cyclopentanoperhydrophenanthrene nucleus) characteristic of the sterols.

The term “estrogens” is art recognized and refers to both natural and synthetic compounds. Natural estrogens are steroid hormones made primarily in the female ovaries and the male testes in humans and other mammals.

The term “progestins” is art recognized and refers to natural or synthetic progestational substance that mimic some or all of the actions of progesterone.

The term “androgens” is art recognized and refers to both natural and synthetic compounds. Natural androgens are steroid hormones made primarily in the male testes in humans and other animals.

The term “anti-inflammatory steroid” or “steroidal anti-inflammatory” is art recognized and refers to a steroid that acts as an anti-inflammatory.

The term “therapeutic effect” is art-recognized and refers to a local or systemic effect in animals, particularly mammals, and more particularly humans caused by a pharmacologically active substance. In other words, the term relates to the effect of any substance intended for use in the diagnosis, cure, mitigation, treatment or prevention of disease or in the enhancement of desirable physical or mental development and/or conditions in an animal or human. The phrase “therapeutically-effective amount” means an amount of such a substance that produces some desired local or systemic effect at a reasonable benefit/risk ratio applicable to any treatment. The therapeutically effective amount of a substance will vary depending upon the subject and disease condition being treated, the weight and age of the subject, the severity of the disease condition, the manner of administration and the like, all of which can readily be determined by one of ordinary skill in the art.

The term “synthetic” is art-recognized and refers to production by in vitro chemical or enzymatic synthesis.

For purposes of this invention, the chemical elements are identified in accordance with the Periodic Table of the Elements, CAS version, Handbook of Chemistry and Physics, 67th Ed., 1986-87, inside cover.

The term “treating” is art-recognized and refers to curing as well as ameliorating at least one symptom of a condition or disease.

The terms “prophylactic” or “therapeutic” treatment are art-recognized and refer to administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if administered after manifestation of the unwanted condition, the treatment is therapeutic (i.e., it is intended to diminish, ameliorate or maintain the existing unwanted condition or side effects therefrom).

A “patient,” “subject” or “host” means either a human or non-human animal.

The term “mammal” is known in the art, and exemplary mammals include humans, primates, bovines, porcines, canines, felines, and rodents (e.g., mice and rats).

The term “bioavailable” is art-recognized and refers to a form of the subject invention that allows for it, or a portion of the amount administered, to be absorbed by, incorporated to, or otherwise physiologically available to a subject or patient to whom it is administered.

The term “poorly-soluble therapeutic agent” refers to a therapeutic agent which is not soluble in a solvent to a critical therapeutic concentration between less than about 3% (w/v).

The term “pharmaceutically acceptable salts” is art-recognized and refers to the relatively non-toxic, inorganic and organic acid addition salts of compounds, including, for example, those contained in compositions of the present invention.

The term “pharmaceutically acceptable excipient” is art-recognized and refers to a pharmaceutically acceptable material, composition or vehicle, such as a liquid or solid filler, diluent, carrier, solvent or encapsulating material, involved in carrying or transporting a subject composition or component thereof from one organ, or portion of the body, to another organ, or portion of the body. Each carrier must be acceptable in the sense of being compatible with the subject composition and its components and not injurious to the patient. Some examples of materials which may serve as pharmaceutically acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) other non-toxic compatible substances employed in pharmaceutical formulations; and (22) water.

The term “adjuvant” is art-recognized and refers to a substance added to a drug that increases its effect. An example of an adjuvant of the instant invention is glycerin.

The term “surfactant” is art-recognized and refers to a material which when used in small amounts modifies the surface properties of liquids or solids. Detergents, wetting agents, emulsifying agents, dispersion agents, and foam inhibitors are all surfactants.

The term “physiologically active substance” is art-recognized and refers to natural, synthetic or genetically engineered chemical or biological compound that is known in the art as having utility for modulating physiological processes in order to afford diagnosis of, prophylaxis against, or treatment of, an undesired existing condition in a living being. Physiologically active substances include drugs such as antianginas, antiarrhythmics, antiasthmatic agents, antibiotics, antidiabetics, antifungals, antihistamines, antihypertensives, antiparasitics, antineoplastics, antitumor drugs, antivirals, cardiac glycosides, herbicides, hormones, immunomodulators, monoclonal antibodies, neurotransmitters, nucleic acids, proteins, radio contrast agents, radionuclides, sedatives, analgesics, steroids, tranquilizers, vaccines, vasopressors, anesthetics, peptides and the like.

Overview

In a preferred embodiment, the invention relates to methods that can be used to develop a liquid formulation in which one or more of the ingredients are in solution. Included are all liquid formulations, including but not limited to solutions, emulsions, suspensions, creams, foams and the like. In certain embodiments, the liquid formulations are suitable for delivery as a spray or aerosol. Liquid formulations of active pharmaceuticals are commercially available; however, some currently available formulations, while safe and effective, are known to precipitate when stored at cool temperatures. Under certain circumstances, the precipitation is reversible, but the FDA believes it is not acceptable to ask consumers to rely upon unstable formulations. High levels of solvents in such formulations will prevent precipitation, but the solvents can cause the sensations of stinging (e.g., when applied to nasal mucosa), poor taste and poor mouth feel; further they must be metabolized and are expensive. However, low levels of solvents can lead to precipitation. This invention relates to a method for determining a level of solvent that will guarantee stability of the formulation at the label storage condition. This invention relates not only to solutions containing active ingredients, but also to those containing inactive ingredients, such as buffers, stabilizers, preservative, antioxidants, thickeners, and the like.

Certain embodiments of this invention relate to pharmaceutical compositions for nasal administration. More particularly, the invention relates to aqueous compositions suitable for nasal administration containing a corticosteroid medicament and methods of development thereof.

Aqueous formulations of anti-inflammatory steroids, such as triamcinolone acetonide, suitable for nasal administration were once commercially available; for example, under the trademark Muro TriNasal® spray. However, currently available formulations, while deemed safe and effective by FDA, are known to precipitate when stored at cool temperatures. One aspect of the invention relates to a method for determining a level of solvent that will provide stability of the formulation over the stated storage conditions. This method has been used to develop a novel formulation of the invention that is suitable for nasal administration of, e.g., anti-inflammatories.

As the temperature of a solution is lowered, a temperature is reached where the solution is saturated with respect to a particular solute. At this point, the amount of material dissolved in the solution is the same as the maximum amount of material that will dissolve in the solution. A further reduction in temperature produces a supersaturated solution. The material dissolved in solution tends to precipitate and form crystals. The first step in forming crystals is the formation of seed crystals. The formation of seed crystals can be slow and it may be necessary to cool the solution 5 to 10 degrees below the temperature where it is saturated. Such a solution is called supersaturated. However, once seed crystals form, the crystals rapidly grow and they will grow at all temperatures wherein the solution is saturated, i.e., not only in supersaturated solutions. In the specific case of Muro TriNasal® spray, the solution is saturated at 25° C., seed crystals form below 20° C. and once seed crystals form, crystallization takes place at any temperature below 25° C.

In general, such crystals cannot be re-dissolved in the formulation by warming the solution because most pharmaceutical products have restricted storage statements, for example 15 to 30° C. In other words, warming above 30° C. is not permitted since high temperature may cause degradation of the formulation.

The study design favored by the Food and Drug Administration and by the ICH Harmonized Tripartite Guideline is to study such products, (solutions, suspensions, semisolids, etc), under accelerated conditions. The products are stored at 40° C. for six months and they are stored 25 and/or 30° C. for the shelf life of the product, generally 18 to 36 months. Such accelerated stability studies or even long-term studies carried out at the upper temperature limit of the label storage condition only serve to mask precipitation problems. The warm temperatures used for such studies prevent crystallization. Further, formulations that form crystals at the lower limit of their storage condition are not detected by these studies.

In addition some products are subjected to a freeze-thaw cycle, e.g., one week stored at minus 20° C., followed by one week at 25° C., followed by one week at minus 20° C. etc. These studies also fail to reveal precipitation problems because the samples spend little time at cool temperatures wherein the sample is in the liquid state. For example, crystals will not form at 25° C. since it is too warm. Further, the crystals will not form at minus 20° C. because the solution has solidified and the molecular motion necessary to form crystals can not occur.

Periodically, samples from these studies are tested for assay and other parameters. This information can be used to predict the chemical stability of solutions. For example, the rate of degradation of the active ingredient can be calculated. From this information, a recommended storage condition can be determined, but it is based only on chemical stability, e.g., the recommended storage condition for Muro TriNasal® spray was 20 to 25° C.

Unfortunately, changes in the physical state, such as precipitation, of the active ingredient or one of the excipients are not always detected by these studies. For example, Muro TriNasal® spray is physically stable at above 25° C. Slightly below 25° C., the concentration of the triamcinolone acetonide in the solution exceeds the solubility limit, i.e., the solution is supersaturated. The triamcinolone acetonide will precipitate when the temperature is several degrees below 25° C., but the precipitation in the absence of seed crystals occurs at such a slow rate that precipitation cannot be observed. However, precipitation will rapidly occur if seed crystals are present.

Muro TriNasal® spray was recalled for low assay. The low assay was caused by precipitation of the active component. The precipitation was caused by short-term exposure to cold temperatures (10 to 20° C.) that produced seed crystals, followed by storage at the label stage condition (20 to 25° C.). Such exposure to cold can occur, for example, in transit by trucks and distribution wharehouses that, e.g., have power failures, resulting in sub-potency issues and therapeutic failure. Since the solution was supersaturated, the crystals continued to grow at 20° C. and the assay decreased until the assay was below the FDA-approved specification. However, as outlined above, if the seed crystals are not present, precipitation does not occur even at 20° C.; and the solution appears to be stable at 20° C. Accordingly, a method is needed to determine the best composition of the vehicle that will prevent supersaturation throughout the label storage conditions. One aspect of the invention relates a method to prevent this type of a stability failure.

Once such method comprises a trial formulation where the active or excipient of interest is supersaturated at temperature close to the storage conditions. The formulation is filled into the same containers that will be used for commercial production. The containers are stored at a temperature below the storage conditions that will promote the formation of seed crystals.

When the desired quantity of seed crystals is present, the containers are emptied and washed to remove free-flowing crystals and the containers are allowed to dry. The containers are filled with several formulations with a range of solvent strengths and the samples are stored at several temperatures. The samples are assayed at intervals and from this data the relationship between temperature, percentage solvent, and solubility can be established. Because the seeds are adhered to the sides of the container, the seeds do not interfere with the analysis. Because the seeds have been formed from a related formulation at a temperature close to the storage conditions, the seeds will be the correct polymorph to seed crystallization from the solution.

This method has been used to develop a formulation where the concentration of the solvent is at the minimum level which will prevent the precipitation and because the level of solvent is as low as possible the formulation will minimize stinging (e.g., when applied to an inflamed nasal mucosa), poor taste, poor mouth feel and the expense of the solvent.

Therapeutic Agents

A vast number of therapeutic agents may be formulated according to the methods of the present invention. In general, therapeutic agents which may be formulated via the methods of the invention include, without limitation: antiinfectives such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

An example of a category of preferred therapeutic agents, that can be used in the present invention, is the steroidal anti-inflammatory drugs. Non-limiting examples of steroidal anti-inflammatory drugs include 21-acetoxypregnenolone, alclometasone, algestone, amcinonide, beclomethasone, beclomethasone dipropionate, betamethasone, budesonide, chloropredinisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluperolone acetate, fluprednidene acetate, fluprednisolone, flurandrenolide, fluticasone propionate, formocortal, halcinonide, halobetasol propionate, halometasone, halopredone acetate hydrocortamate, hydrocortisone, loteprednol etabonate, mazipredone, medrysone, meprednisone, methylprednisolone, mometasone furoate, mometasone furoate monohydrate, paramethasone, prednicarbate, prednisolone, prednisolone 25-diethylaminoacetate, prednisolone sodium phosphate, prednisone, prednival, prednylidene rimexolone, tixocortol, triamcinolone, triamcinolone acetonide, triamcinolone benetonide, and triamcinolone hexacetonide.

Another preferred example of a category of therapeutic agents that can be used in the present invention are antimicrobial drugs. Non-limiting examples of antimicrobial drugs include salts of lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole and amanfadine.

A preferred example of a category therapeutic agents that can be used in the present invention are antibiotics drugs. Non-limiting examples of antibiotics drugs include aminocillin, amikacin, amoxicillin, amoxicillin and clavulanate, ampicillin, azlocillin, aztreonam, bacampicillin, carbenicillin, cefaclor, cefadroxil, cefamandole, cefazolin, cefonicid, cefoperazone, ceforanide, cefotaxime, cefotetan, cefoxitin, ceftazidime, ceftibuten, ceftizoxime, ceftriaxone, cefuroxime, cephalexin, cephalothin, cephapirin, cephradine, chloramphenicol, cinoxacine, clindamycin, cloxacillin, cyclacillin, cycloserine, demeclocycline, dicloxacillin, doxycycline, erythromycin, erythromycin and sulfisoxazole, flucloxacillin, fusidic acid, gentamicin, imipenem and cilastatin, kanamycin, lincomycin, methacycline, methenamine, methicillin, metronidazole, mezlocillin, minocycline, moxalactam, nafcillin, nalidixic acid, netilmicin, nitrofurantoin, norfloxacin, oxacillin, oxytetracycline, penicillin G, penicillin V, piperacillin, pivampicillin, rifabutin, rifampin, spectinomycin, streptomycin, sulfacytine, sulfadiazine and trimeth, oprim, sulfamethoxazole, sulfamethoxazole, trimethoprim, sulfisoxazole, tetracycline, ticarcillin, ticarcillin, clavulanate, tobramycin, trimethoprim, and vancomycin.

In addition, another preferred example of a category therapeutic agents that can be used in the present invention is antifungals drugs. Non-limiting examples of antifungal drugs include azoles such as clotrimazole (Mycelex® or Lotrimin®), enconazole (Spectazole®), miconazole, fluconazole (Diflucan®), itraconazole (Sporanox®), ketoconazole (Nizoral®), griseofulvin and related compounds, polyenes including amphotericines and nystatin, terbinafine (Lamisil®), butenafine (Mentax®), ciclopirox (Loprox®), and tolnaftate (Tinactin®).

Non-steroidal anti-inflammatory agents (NSAIDS) may also be formulated by the methods of the invention. Non-limiting examples of NSAIDS include propionic acid derivatives, acetic acid, fenamic acid derivatives, biphenylcarboxylic acid derivatives, oxicams, including but not limited to aspirin, acetaminophen, ibuprofen, naproxen, benoxaprofen, flurbiprofen, fenbufen, ketoprofen, indoprofen, pirprofen, carporfen, and bucloxic acid.

Steroidal hormones (glucocorticoids, mineralocorticoids, androgens, estrogens and progestins) may also be formulated by the methods of this invention. Non-limiting examples of steroid hormones includes corisol, aldosterone, testosterone, dehydroepiandrosterone, sehydroepiandrosteron sulfate, androstenedione, dihydrotestosterone, estradiol, estrone, estriol, progesterone, prednisone, dexamethasone, triamcinolone, flusrocortisone, oxandrolone, decadurabolin and other anabolic steriods, diethylstilbestrol, norethindrone and medoxyprogesterone acetate.

Antihistamines may also be formulated by the methods of this invention. Non-limiting examples of antihistamines include adrenocorticoids, glucocorticoid, albuterol, aminophylline, astemizole, beclomethasone, bitolterol, budesonide, cetirizine, corticotropin, cromolyn, dexamethasone, dyphylline, ephedrine, epinephrine, ethylnorepinephrine, fenoterol, flunisolide, ipratropium, isoetharine, isoproterenol, isoproterenol, phenylephrine, loratadine, metaproterenol, oxtriphylline, oxtriphylline, guaifenesin, pirbuterol, racepinephrine, terbutaline, terfenadine, theophylline, theophylline, guaifenesin, and triamcinolone.

Optional Components

It will be recognized by those skilled in the art that for many pharmaceutical compositions it is usual to add at least one antioxidant to prevent degradation and oxidation of the pharmaceutically active ingredients. It will also be understood by those skilled in the art that colorants, flavoring agents and non-therapeutic amounts of other compounds may be included in the formulation. Examples of flavoring agents are menthol and other fruit flavors.

The formulations of the present invention may include ancillary agents, for example a pH-buffering system, preferably a buffer such as phosphate, citrate or acetate buffers, a preservative and an osmotic pressure controlling agent, e.g. glycerol or sodium chloride.

The concentration of the active agent in the preparations of this invention will depend on the particular agent chosen, on its efficacy, on a comparison of its bioavailability by nasal administration and by other routes of administration, for example parenteral injection, and on the desired frequency of administration combined with the desired single dosage of the formulation. Such pharmacological data can routinely be obtained by the skilled artisan from animal experiments.

The formulation of the present invention may contain, in addition to the active agent, one or more other active substances such as a non-steroidal antiinflammatory agent (e.g., mefenamic acid), an antihistaminic (e.g., clemastine fumarate, terfenadine, chlopheniramine maleate, or diphenhydramine hydrochloride), an antibiotic (e.g., dirithromycin, erythromycin, or tetracycline), an antifungal agent (e.g., miconazole), and/or an antimicrobial agent (e.g., sulfamethizole, sulfamethoxazole, or sulfisoxazole), each in a suitable amount.

A formulation according to the present invention may further contain other pharmacologically active substances, such as a vasoconstrictor, a surface anesthetic, etc., in suitable amounts. The vasoconstrictor includes but is not limited to naphazoline nitrate and phenylephrine hydrochloride. The surface anesthetic includes but is not limited to lidocaine, lidocaine hydrochloride, and mepivacaine hydrochloride. These pharmacologically active substances are used in a proportion of generally about 0.01 to about 10 w/w % and preferably about 0.05 to about 5 w/w %.

A formulation according to the present invention may contain various additives which are broadly used in nasal drops in general. Among such additives are preservatives, isotonizing agents, buffers, stabilizers, pH control agents, and suspending agents. The preservative that can be used includes parabens (e.g. methyl p-hydroxybenzoate, propyl p-hydroxybenzoate, etc.), invert soaps (e.g. benzalkonium chloride, benzethonium chloride, chlorhexidine gluconate, cetylpyridinium chloride, etc.), alcohol derivatives (e.g. chlorobutanol, phenethyl alcohol, etc.), organic acids (e.g. dehydroacetic acid, sorbic acid, etc.), phenols (e.g. p-chloromethoxyphenol, p-chlorometacresol, etc.), and organomercury compounds (e.g. thimerosal, phenylmercury nitrate, nitromersol, etc.). The isotonizing agent includes but is not limited to glycerin, propylene glycol, sorbitol, and mannitol. The buffer that can be used includes boric acid, phosphoric acid, acetic acid, and amino acids, among others. The stabilizer includes antioxidants (e.g. dibutylhydroxytoluene (BHT), butylhydroxyanisole (BHA), propyl gallate, etc.), and chelating agents (edetic acid, citric acid, etc.). The pH control agent includes hydrochloric acid, acetic acid, sodium hydroxide, phosphoric acid, citric acid, etc. As the suspending agent, various surfactants (nonionic surfactants such as polysorbate 80, polyoxyethylene hydrogenated castor oil, tyloxapol; cationic surfactants such as quaternary ammonium salts; anionic surfactants such as alkylsulfates; and amphoteric surfactants such as lecithin) can be employed.

Administration

The pharmaceutical formulations of this invention can be administered to humans and other mammals orally, rectally, parenterally, intracistemally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrastemal, subcutaneous and intraarticular injection and infusion.

Administration: Inhalation Delivery

In a preferred embodiment, the active agent in the stable compositions of the present invention may be any compound capable of oral or nasal inhalation delivery. The preparations of this invention may be used in any dosage dispensing device adapted for intranasal administration. The device should be constructed with a view to ascertaining optimum metering accuracy and compatibility of its constructive elements, such as container, valve and actuator with the nasal formulation and could be based on a mechanical pump system, e.g., that of a metered-dose nebulizer, or on a pressurized aerosol system. The aerosol system requires the propellant to be inert towards the formulation. Suitable propellants may be selected among such gases as fluorocarbons, hydrocarbons, nitrogen and dinitrogen oxide or mixtures thereof. In addition, irrigating devices such as syringes or water pics may be used.

The inhalation delivery device can be a nebulizer or a metered dose inhaler (MDI), or any other suitable inhalation delivery device known to one of ordinary skill in the art. The device can contain and be used to deliver a single dose of the active agent compositions or the device can contain and be used to deliver multi-doses of the compositions of the present invention.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device may comprise a nozzle that has two coincident outlet channels of defined aperture size through which the liquid formulation can be accelerated. This results in impaction of the two streams and atomization of the formulation. The nebulizer may use a mechanical actuator to force the liquid formulation through a multi-orifice nozzle of defined aperture size(s) to produce an aerosol of the formulation for inhalation. In the design of single dose nebulizers, blister packs containing single doses of the formulation may be employed.

In the present invention the nebulizer may be employed to ensure the sizing of particles is optimal for positioning of the particle within, for example, the mucous membrane.

A metered dose inhalator (MDI) may be employed as the inhalation delivery device for the compositions of the present invention. This device is pressurized (pMDI) and its basic structure consists of a metering valve, an actuator and a container. A propellant is used to discharge the formulation from the device. The composition may consist of particles of a defined size suspended in the pressurized propellant(s) liquid, or the composition can be in a solution or suspension of pressurized liquid propellant(s). The propellants used are primarily atmospheric friendly hydroflurocarbons (HFCs) such as 134a and 227. Traditional chloroflurocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To insure accuracy of dosing, the delivery of the formulation may be programmed via a microprocessor to occur at a certain point in the inhalation cycle. The MDI may be portable and hand held.

Administration: Ophthalmic and Otic Formulations

Alternatively, the active agent in the stable compositions of the present invention may be any compound capable of being delivered to the eye or to the ear. The preparation of ophthalmic and otic solutions requires consideration of factors such as the inherent toxicity of the drug itself, isotonicity value, the need for buffering agents, and the need for a preservative. Ophthalmic solutions are sterile solutions, essentially free from foreign particles, suitably compounded and packaged for instillation into the eye. Preparation of ophthalmic solution requires careful consideration of such factors as the inherent toxicity of the drug itself, isotonicity value, the need for buffering agents, and the need for preservatives. Ideally, an ophthalmic solution would have an isotonicity value of about 0.9% sodium chloride, but the eye can tolerate isotonicity values as low as that of about 0.6% sodium chloride and as high as that of about 2.0% sodium chloride. Some ophthalmic solutions are necessarily hypertonic in order to enhance absorption and provide a concentration of active ingredient(s) strong enough exert a prompt and effective action. An ophthalmic preparation with a buffer system approaching physiological pH is ideal. Similar considerations need also be made for nasal and otic products. Otic solutions, often intended for instillation in the outer ear, are aqueous or they are solutions prepared with glycerin or other solvents and dispersing agents.

Administration: Topical Formulations

In another preferred embodiment, the active agent in the stable compositions of the present invention may be any compound capable of being delivered via a topical formulation. The topical formulations of the invention may take the form of, e.g., a lotion or cream, that is, those formulations which include a relatively large aqueous phase and a relatively small oil phase. Furthermore, the lotions and creams of the invention may include the active component “all-in-solution” in the oil phase so that substantially no steroid crystallizes out at room temperature. Alternatively, the lotion or cream may comprise a biphasic system, that is, a system wherein a portion (from about 30 to about 75% by weight) of the active agent is in solution in the oil phase and the remainder of the active agent is in suspension in the aqueous phase.

With regard to the cream formulations of the invention, wherein all of the active agent is in solution, the cream will contain less than about 10% by weight of the active ingredient based on the weight of the entire cream formulation. In certain embodiments, the cream will contain from about 0.005 to about 0.6% and preferably from about 0.025 to about 0.2% by weight of the active ingredient based on the weight of the entire cream formulation. The all-in-solution cream formulation will also include in the oil phase, in addition to the active agent, from about 5 to 14% and preferably from about 8 to about 12% by weight of the emulsifier-thickener based on the weight of the entire cream formulation, and from about 2 to about 8% and preferably from about 3 to about 5% by weight of oleaginous material or emollient based on the weight of the entire cream formulation. The oil phase may also optionally include an anti-whitening agent or anti-foaming agent in an amount within the range of from about 0.2 to about 3% and preferably from about 0.5 to about 1.5% by weight based on the entire cream formulation. An antioxidant may also optionally be included in an amount within the range of from about 0.005 to about 0.04% and preferably from abut 0.01 to about 0.03% by weight based on the entire cream formulation.

The aqueous phase of the all-in-solution cream formulation will contain a glycol-type preservative such as propylene glycol in an amount within the range of from about 10 to about 50% and preferably from about 12 to about 40% by weight of the entire cream formulation and/or a paraben or other conventional type preservative such as methyl and/or propyl paraben in an amount ranging from about 0.05 to about 0.5%, and purified water in an amount within the range of from about 30 to about 85% by weight and preferably from about 35 to about 65% by weight of the entire cream formulation.

With regard to the cream formulation of the invention in the form of the biphasic system, the cream will contain from about 0.6% and preferably from about 0.025 to about 0.2% by weight of the active ingredient based on the weight of the entire cream formulation. The biphasic cream formulation will also include in the oil phase, in addition to the active agent, from about 8 to about 12% and preferably from about 9 to about 11% by weight of the emulsifier-thickener based on the weight of the entire cream formulation, and from about 2 to about 8% and preferably from about 3 to about 6% by weight of oleaginous material or emollient based on the weight of the entire cream formulation. The oil phase may also optionally include an anti-whitening agent or anti-foaming agent in an amount within the range of from about 0.2 to about 3% and preferably from about 0.5 to about 1.5% by weight based on the entire cream formulation. An antioxidant may also optionally be included in an amount within the range of from about 0.05 to about 0.04% and preferably from about 0.01 to about 0.03% by weight based on the entire cream formulation.

The aqueous phase of the biphasic cream formulation will contain a preservative in amount within the range of from about 10 to about 50% and preferably from about 12 to about 40% by weight of the entire cream formulation, and purified water in an amount within the range of from about 30 to about 85% by weight and preferably from about 35 to about 65% by weight of the entire cream formulation.

With regard to the lotion formulation of the invention where the active agent is to be all-in-solution, the lotion will contain from about 0.005 to about 0.6% and preferably from about 0.025 to about 0.2% by weight of the active ingredient based on the weight of the entire lotion formulation. The all-in-solution lotion formulation will also include in the oil phase, in addition to the active agent, from about 5 to about 14% and preferably from about 8 to about 12% by weight of the emulsifier-thickener based on the weight of the entire lotion formulation, and from about 0.5 to about 6% and preferably from about 1 to about 5% by weight of oleaginous material or emollient based on the weight of the entire lotion formulation. The oil phase may also optionally include an anti-whitening agent or anti-foaming agent in an amount within the range of from about 0.2 to about 3% and preferably from about 0.5 to about 1.5% by weight based on the entire lotion formulation. An antioxidant may also optionally be included in an amount within the range of from about 0.005 to about 0.04% and preferably from about 0.01 to about 0.03% by weight based on the entire lotion formulation.

The aqueous phase of the all-in-solution lotion formulation will contain glycol-type preservative in an amount within the range of from about 10 to about 50% an preferably from about 12 to about 40% by weight of the entire lotion formulation, and/or a paraben or other conventional type preservative in amount ranging from about 0.05 to about 0.5%, and purified water in an amount within the range of about 50 to about 90% by weight an preferably from about 60 to about 85% by weight of the entire lotion formulation.

With regard to the biphasic lotion formulation of the invention, the lotion will contain from about 0.005 to about 0.6% and preferably from about 0.025 to about 0.2% by weight of the active ingredient based on the weight of the entire lotion formulation. The biphasic lotion formulation will also include in the oil phase, in addition to the active agent, from about 1 to about 5% and preferably from about 2 to about 4% by weight of the emulsifier-thickener based on the weight of the entire lotion formulation, and from about 0.2 to about 5% and preferably from about 0.5 to about 4% by weight of oleaginous material or emollient based on the weight of the entire lotion formulation. The oil phase may also optionally include an anti-whitening agent or anti-foaming agent in an amount within the range of from about 0.2 to about 3% and preferably from about 0.5 to about 1.5% by weight based on the entire lotion formulation. An antioxidant may also optionally be included in an amount within the range of from about 0.005 to about 0.04% and preferably from about 0.01 to about 0.03% by weight based on the entire lotion formulation.

The aqueous phase of the biphasic lotion formulation will contain a glycol-type preservative such as propylene glycol in an amount within the range of from about 8 to about 50% and preferably from about 10 to about 40% by weight of the entire lotion formulation, and/or paraben-type or other preservatives at their recommended amount as described above, and purified water in an amount within the range or from about 50 to about 90% by weight and preferably from about 60 to about 85% by weight of the entire lotion formulation.

Suitable thickeners include those conventionally employed in topical creams such as, for example, monoglycerides and fatty alcohols, fatty acid esters of alcohols having from about 3 to about 16 carbon atoms. Examples of suitable monoglycerides are glyceryl monostearate and glyceryl monopalmitate. Examples of fatty alcohols are cetyl alcohol and stearyl alcohol. Examples of suitable esters are myristyl stearate and cetyl stearate. The monoglyceride also functions as an auxiliary emulsifier. Other emollients or oleaginous material which may be employed include petrolatum, glyceryl monooleate, myristyl alcohol and isopropyl palmitate.

Dosages

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions (e.g., the steroidal anti inflammatory drug) because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

In general, the doses of an active agent will be chosen by a physician based on the age, physical condition, weight and other factors known in the medical arts.

Efficacy of Treatment

The efficacy of treatment with the subject compositions may be determined in a number of fashions known to those of skill in the art.

In one exemplary method, the median rate of decrease in inflammation for treatment with a subject composition may be compared to other forms of treatment with the particular anti inflammatory steroid contained in the subject composition, or with other anti inflammatory steroid. The decrease in inflammation for treatment with a subject composition as compared to treatment with another method may be 10, 25, 50, 75, 100, 150, 200, 300, 400% greater or even more. The period of time for observing any such decrease may be about 1, 3, 5, 10, 15, 30, 60 or 90 or more hours. The comparison may be made against treatment with the particular anti inflammatory steroid contained in the subject composition, or with other anti inflammatory steroid, or administration of the same or different agents by a different method, or administration as part of a different drug delivery device than a subject composition. The comparison may be made against the same or a different effective dosage of the various agents.

Alternatively, a comparison of the different treatment regimens described above may be based on the effectiveness of the treatment, using standard indices for inflammation known to those of skill in the art. One method of treatment may be 10%, 20%, 30%, 50%, 75%, 100%, 150%, 200%, 300% more effective, than another method.

Alternatively, the different treatment regimens may be analyzed by comparing the therapeutic index for each of them, with treatment with a subject composition as compared to another regimen having a therapeutic index two, three, five or seven times that of, or even one, two, three or more orders of magnitude greater than, treatment with another method using the same or different anti inflammatory steroid.

Kits

This invention also provides kits for conveniently and effectively implementing the methods of this invention. Such kits comprise any subject composition, and a means for facilitating compliance with methods of this invention. Optionally, the aqueous formulation of the kit further comprises an antihistamine, decongestant, ophthalmological, antibiotic, mucolytic agents, antifungals or irrigating solution. The present invention also relates to any of the aforementioned kits, further comprising a solid or liquid dosage form of an antihistamine, decongestant, ophthalmological, or antibiotic. The present invention also relates to any of the aforementioned kits, further comprising a separate irrigating solution.

Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, the invention contemplates a kit including compositions of the present invention, and optionally instructions for their use. An aqueous formulation contained in a kit of the present invention may further comprise an antihistamine, decongestant, ophthalmological, antibiotic, mucolytic agent or irrigating solution.

### EXEMPLIFICATION

Recalled Muro TriNasal® Spray

Production lots of Muro TriNasal® spray were recalled by FDA because the active ingredient had precipitated during long-term storage in Muro retention areas at temperatures occasionally below the label storage temperature of 20-25° C. Therefore, a non-expired lot of recalled Muro TriNasal® spray was tested for stability. The formulation is an aqueous propylene glycol solution of triamcinolone acetonide. The viscosity enhancing agent is polyethylene glycol 3350.

Fifteen mL of the formulation was transferred into a 20 mL amber PET bottle using a Valois metered-dose pump. Three different lots were assayed for triamcinolone acetonide concentration. Lot 10808A assayed 91.5% for triamcinolone acetonide, meaning that the observed concentration of triamcinolone acetonide was only 91.5% of the concentration stated on the label. The limits are: 90.0 to 110.0%. Additional lots of Muro TriNasal® spray were tested. Of 18 lots, four lots showed triamcinolone acetonide levels at or below the 90% specification limit. The lots had been stored at conditions outside the label storage statement. The label storage condition is 20-25° C. However, product was stored at temperatures as low as 16.5° C. Microscopic examination of the Muro TriNasal® spray bottles revealed crystals of triamcinolone acetonide. Long-term storage samples that were stored at 25° C. assayed 100%. Accordingly, the triamcinolone acetonide appeared to have crystallized out of solution due to storage at temperatures below the range described in the label-storage statement.

### Example 1

Retention samples of Muro TriNasal® spray (lot 10605), were subsequently stored in 10, 15, 20, 25 and 30° C. environmental chambers. Samples were periodically assayed. Since the label storage condition is 20° to 25° C., it was expected that the triamcinolone acetonide in those samples stored at 20° C. and above would re-dissolve and the assay would return to 100%. The results are shown below in Table 1:

TABLE 1
Time and temperature stability results for recalled Muro TriNasal ® spray
(lot 10605; expiry date, November 2002) subsequently stored in environmental
chambers.

| | Conc | Conc | Conc | Conc | Conc | Conc | |
|---|---|---|---|---|---|---|---|
| | When | When | When | When | When | When | (% change |
| Storage | Stored for | Stored for | Stored for | Stored for | Stored for | Stored for | in concentration)/ |
| Temp. | 4 days | 7 days | 14 days | 35 days | 65 days | 95 days | month |
| 30 C. | 88.6% | 90.2% | 93.0% | 90.0% | 96.6% | 101.2% | 3.7% |
| 25 C. | 87.2% | 87.8% | 88.4% | 84.6% | 91.4% | 93.2% | 1.93% |
| 20 C. | 86.4% | 88.2% | 88.8% | 84.8% | 89.8% | 90.7% | 0.04% |
| 15 C. | 88.0% | 87.4% | 87.2% | 82.0% | 87.0% | 87.5% | −0.05% |
| 10 C. | 84.8% | 86.6% | 86.6% | 79.6% | 78.0% | 76.8% | −3.35% |

At 25° and 30° C. the triamcinolone acetonide assay increased with time. At 10° and 15° C. the triamcinolone acetonide decreased with time. The samples stored at 20° C. did not change. The fact that the assay for the 20° C. samples did not increase with time suggests that the recalled Muro TriNasal® spray formulation may not be stable with respect to precipitation of the triamcinolone acetonide over the label storage condition.

### Example 2

### Propylene Glycol Content of Nasal Formulations

The composition of the FDA-approved Muro TriNasal® spray formulation is shown below in Table 2.

TABLE 2
Recalled Muro TriNasal ® spray formulation (triamcinolone acetonide).

| Components | Function | g/100 mL |
|---|---|---|
| Triamcinolone acetonide USP | Active Ingredient | 0.05 |
| Propylene glycol USP | Solvent | 12.00 |
| Polyethylene glycol 3350 | Viscosity Enhancing agent | 40.00 |
| Edetate disodium USP | Chelating agent | 0.050 |
| Citric acid USP | Buffer | 0.72 |
| Sodium citrate | Buffer | 0.74 |
| 50% Benzalkonium chloride | Preservative | 0.020 |
| USP | | |
| Purified water | Vehicle | 54.42 |

The solubility of triamcinolone acetonide can be increased by increasing the concentration of propylene glycol in the formulation. As presented below in Table 3, five formulations with varying percentages of propylene glycol were prepared and tested for stability. Formulation 3267001 has the same percentage of propylene glycol as the FDA-approved product, Muro TriNasal® spray. The other four formulations have greater percentages of propylene glycol. The percentage of polyethylene glycol in these formulations was decreased slightly to keep the viscosity approximately constant. The concentration of the remaining ingredients was held constant.

TABLE 3
Percentage of propylene glycol in samples.

| Formulation number | Propylene glycol (g/100 mL) |
|---|---|
| 3267001 | 12.00 |
| 3267002 | 13.00 |
| 3267003 | 14.00 |
| 3267004 | 16.00 |
| 3267005 | 20.00 |

Each of the above formulations was filled into 20 mL amber polyethylene terephthalate bottles. The fill volume was 15 ml. Bottles were capped with Valois VP7/90 pumps. The components were taken from retention samples of the product which contained seed crystals. Expired retention samples were emptied, the components were washed with water to remove the product and the components were air dried. Crystals of triamcinolone acetonide are attached to the inside surfaces of the bottles. These crystals can be seen under a microscope. The seed crystals initiate crystallization of supersaturated solutions.

Samples were placed in 10, 15, 20, 25 and 30° C. chambers and tested for triamcinolone acetonide. If the vehicle is not saturated with triamcinolone acetonide at a giving storage temperature, the concentration will increase as the seeds dissolve. If the vehicle is super-saturated at a giving storage temperature, the concentration will decrease as the triamcinolone acetonide comes out of solution and the crystals grow. The results are presented below in Table 4.

TABLE 4
Relative Amounts of Triamcinolone Acetonide in Nasal Formulations as a
Function of Propylene Glycol Content and Long-Term Storage Temperature.

| % Propylene Glycol | Temp | | t = one | t = two | t = four | t = six | t = eight |
|---|---|---|---|---|---|---|---|
| (Formulation Number) | (C.) | t = 0 | week | weeks | weeks | weeks | weeks |
| 12% PG (3267001) | 30 | | 98.1 | 99.9 | 102.5 | 101.5 | 106.5 |
| 12% PG (3267001) | 25 | 97.2 | 96.4 | 97.0 | 97.1 | 96.0 | 101.4 |
| 12% PG (3267001) | 20 | | 95.1 | 91.1 | 87.9 | 83.7 | 87.2 |
| 12% PG (3267001) | 15 | | 92.6 | 85.0 | 76.0 | 73.5 | 74.9 |
| 12% PG (3267001) | 10 | | 88.6 | 75.5 | 65.8 | 59.4 | 60.9 |
| 13% PG (3267002) | 30 | | 97.4 | 99.3 | 101.6 | 99.5 | 106.7 |
| 13% PG (3267002) | 25 | 96.8 | 96.8 | 97.5 | 99.6 | 96.4 | 102.6 |
| 13% PG (3267002) | 20 | | 95.5 | 93.9 | 93.0 | 89.6 | 94.6 |
| 13% PG (3267002) | 15 | | 93.0 | 89.5 | 80.3 | 79.1 | 81.0 |
| 13% PG (3267002) | 10 | | 85.6 | 73.6 | 64.9 | 62.4 | 63.7 |
| 14% PG (3267003) | 30 | | 98.5 | 101.3 | 103.0 | 101.1 | 106.4 |
| 14% PG (3267003) | 25 | 97.2 | 97.2 | 97.9 | 98.0 | 97.4 | 102.7 |
| 14% PG (3267003) | 20 | | 96.6 | 97.3 | 95.6 | 95.3 | 100.9 |
| 14% PG (3267003) | 15 | | 96.1 | 95.0 | 89.9 | 87.3 | 88.9 |
| 14% PG (3267003) | 10 | | 95.2 | 90.1 | 77.1 | 74.9 | 72.0 |
| 16% PG (3267004) | 30 | | 98.8 | 99.1 | 94.9 | 101.3 | 107.5 |
| 16% PG (3267004) | 25 | 97.0 | 97.3 | 97.2 | 95.3 | 99.5 | 105.5 |
| 16% PG (3267004) | 20 | | 96.5 | 96.9 | 91.9 | 96.3 | 102.7 |
| 16% PG (3267004) | 15 | | 96.6 | 95.9 | 91.8 | 95.7 | 102.0 |
| 16% PG (3267004) | 10 | | 96.1 | 95.9 | 90.6 | 93.6 | 96.7 |
| 20% PG (3267005) | 30 | | 101.1 | 95.4 | 92.1 | 103.4 | 109.1 |
| 20% PG (3267005) | 25 | 92.8 | 97.9 | 93.5 | 90.9 | 98.1 | 105.0 |
| 20% PG (3267005) | 20 | | 96.9 | 92.7 | 90.2 | 98.4 | 107.7 |
| 20% PG (3267005) | 15 | | 96.6 | 90.0 | 88.8 | 98.4 | 106.3 |
| 20% PG (3267005) | 10 | | 96.3 | 92.8 | 88.1 | 94.9 | 100.5 |

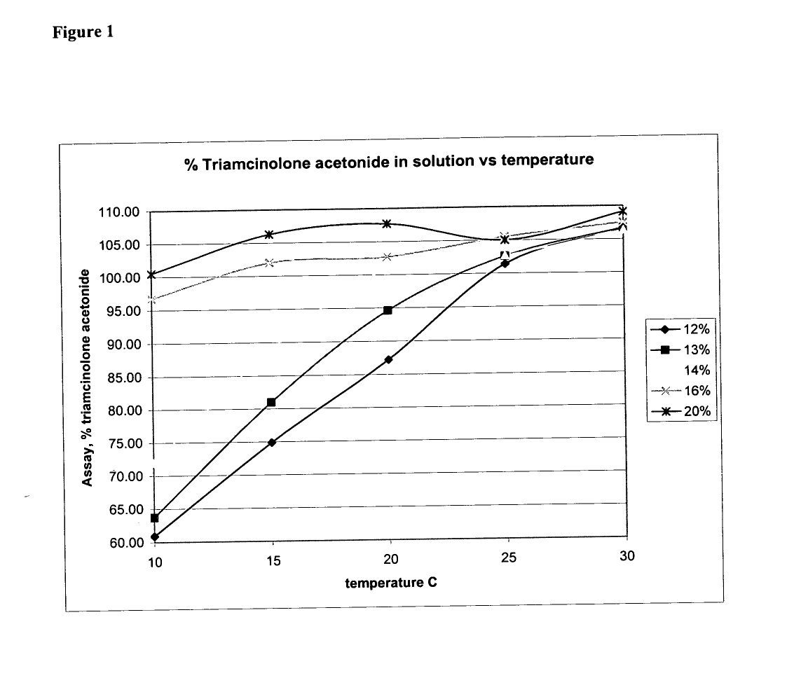

The label storage condition is 20° to 25° C. The assay for the 2 month 20° C. samples should be 100%. The 2 month 20° C. assay for the formulation, 3267003, 14% PG is 100.9%. The formulation containing 14% PG, propylene glycol, is stable at 20° C. The formulations with more than 14% PG are also stable. These results are plotted in FIG. 1.

The triamcinolone acetonide is plotted versus temperature for the five formulations. The bottom curve is the data for the formulation containing 12% propylene glycol; this is the formulation approved in NDA 12-120. This formulation is stable down to 25° C. Below that temperature the solution is supersaturated and the triamcinolone will crystallize out. For each formulation there is a temperature where the solution is supersaturated. These temperatures are presented in Table 5.

TABLE 5
Super saturation temperature for the various propylene glycol levels.

| Formulation, % propylene glycol | Super saturation temperature |
|---|---|
| 12% (3267001) | 25° C. |
| 13% (3267002) | 23° C. |
| 14% (3267003) | 20° C. |
| 16% (3267004) | 13° C. |
| 20% (3267005) | Below 10° C. |

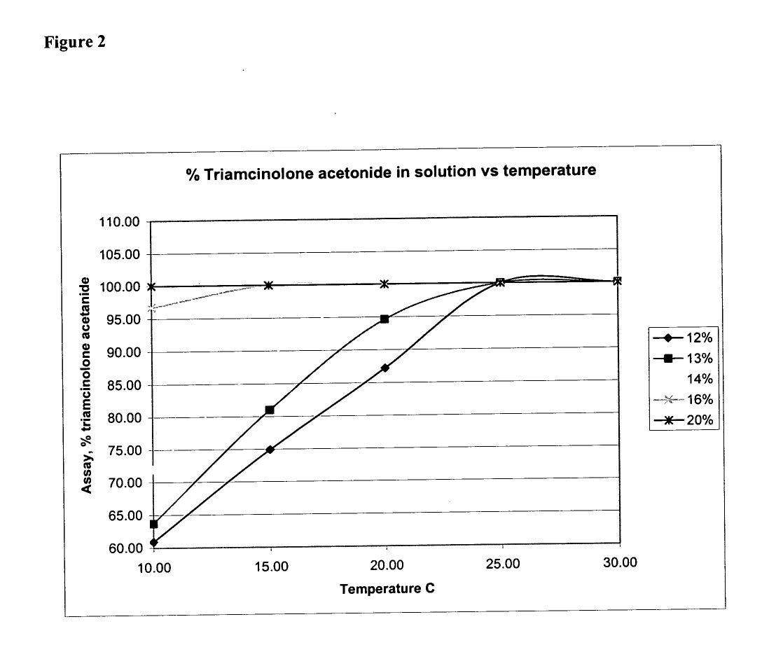

There are two events happening in these studies. Below the super saturation temperature, triamcinolone acetonide is crystallizing onto the seed crystals. Above the super saturation temperature, triamcinolone acetonide seed crystals are dissolving and the assay is increasing above 100%. In FIG. 2, the results are presented in a graph where all assays above 100% have been truncated to 100%. This graph more clearly shows the behavior of these formulations.

### Incorporation by Reference

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

1. An aqueous formulation, comprising:
water;
a therapeutic agent, selected from the group consisting of anti-inflammatory steroids and steroidal hormones, in an amount between about 0.001% and about 2.0% (w/v);
propylene glycol in an amount between about 13% and about 20% (w/v);
polyethylene glycol (PEG) in an amount between about 10% and about 50% (w/v);
a preservative;
a stabilizer; and
a pH buffering agent sufficient to maintain the pH of the aqueous formulation at between about 3.5 and about 8.0.

2. The aqueous formulation of claim 1, wherein the amount of propylene glycol is about 14% (w/v).

3. The aqueous formulation of claim 1, wherein the amount of preservative is between about 0.01% and about 0.08% (w/v).

4. The aqueous formulation of claim 1, wherein the amount of stabilizer is between about 0.005% and about 0.05% (w/v).

5. The aqueous formulation of claim 1, wherein the preservative is benzalkonium chloride.

6. The aqueous formulation of claim 1, wherein the stabilizer is disodium ethylenediaminetetraacetic acid (EDTA).

7. The aqueous formulation of claim 1, wherein the therapeutic agent is a steroidal hormone selected from the group consisting of benzestrol, broparoestriol, chlorotrianisene, clopormon, desogesterol, dienestriol, equilenin, equilin, estradiol, estriol, estrone, ethinyl estradiol, gestodene, hexestrol, lynestrenol, mestranol, methallenestril, methestrol, moxestriol, mytatrienediol, norethindrone, norethynodrel, norgestimate, quinestradiol, quinestrol, allylestrenol, altrenogest, anagestone, chlormadinone acetate, delmadinone acetate, demegestone, dimethisterone, drospirenone, dydrogesterone, ethisterone, ethynodiol, flurogestone acetate, gestonorone caproate, 17-hydroxy-16-methylene-X⁶-progesterone, 17α-hydroxyprogesterone, medrogestone, medroxyprogesterone, megestrol acetate, melengestrol, norgesterone, norgestrel, norgestrienone, norvinisterone, pentagesterone, progesterone, promegestrone, trengestrone, boldenone, cloxotestosterone, fluoxymesterone, mesterolone, methandrostenolone, 17-methyltestosterone, 17α-methyltestosterone-3-cylcopentyl enol ether, mibolerone, norethandrolone, normethandrone, oxandrolone, oxyrnesterone, oxymetholone, stanolone, stanozolol, testosterone, tismesterone, and mixtures of any of them.

8. The aqueous formulation of claim 1, wherein the therapeutic agent is an anti-inflammatory steroid selected from the group consisting of 21-acetoxypregnenolone, alclometasone, algestone, alisactide, amcinonide, aminoglutethimide, beclomethasone, beclomethasone dipropionate, betamethasone, betamethasone dipropionate, betamethasone adamantoate, budesonide, butixocort, chloropredinisone, ciclometasone, clobetasol, clobetasone, clocortolone, cloprednol, corticosterone, cortisone, cortivazol, deflazacort, deprodone, deprodone propionate, desonide, desoximetasone, dexamethasone, dexamethasonisonicotinate, diflorasone, diflucortolone, difluprednate, endrisone, enoxolone, fluazacort, flucloronide, flumethasone, flunisolide, fluocinolone acetonide, fluocinonide, flucortin, fluocortin butyl, flodexan fluclorolone acetonide, fluocortolone, fluorometholone, fluperolone acetate, fluprednidene acetate, fluprednisolone, flurandrenolide, fluticasone propionate, formebolone, formocortal, halcinonide, halobetasol propionate, halometasone, halopredone acetate hydrocortamate, hydrocortisone, hydrocortisone aceponate, hydrocortisone butyrate, hydrocortisone-17-butyrate, icomethasone enbutyate, loteprednol etabonate, lotrisone, mazipredone, medrysone, meprednisone, methylprednisolone, mometasone furoate, mometasone furoate monohydrate, mycophenolate mofetil, paramethasone, pranlukast, prednicarbate, prednisolone, prednisolone 25-diethylaminoacetate, prednisolone sodium phosphate, prednisone, prednival, prednylidene promedrol, rimexolone, seratrodast, tipredane, tixocortol, triamcinolone, triamcinolone acetonide, triamcinolone benetonide, triamcinolone hexacetonide, triiostane, ulobetasol propionate, zileuton, and mixtures of any of them.

9. The aqueous formulation of claim 1, wherein the therapeutic agent is triamcinolone acetonide.

10. The aqueous formulation of claim 9, wherein the amount of propylene glycol is about 14% (w/v).

11. The aqueous formulation of claim 9, wherein the amount of preservative is between about 0.01% and about 0.08% (w/v).

12. The aqueous formulation of claim 9, wherein the amount of stabilizer is between about 0.005% and about 0.05% (w/v).

13. The aqueous formulation of claim 9, wherein the preservative is benzalkonium chloride.

14. The aqueous formulation of claim 9, wherein the stabilizer is disodium ethylenediaminetetraacetic acid (EDTA).

15. An aqueous formulation, comprising:
water;
triamcinolone acetonide in an amount between about 0.01% and about 0.05% (w/v);
propylene glycol in an amount of about 14% (w/v);
PEG in an amount between about 35% and 45% (w/v);
benzalkonium chloride in an amount of about 0.05% (w/v);
disodium EDTA in an amount of about 0.05% (w/v);
citric acid in an amount of about 0.72% (w/v);
sodium citrate dihydrate in an amount of about 0.74% (w/v); and
an amount of a pH buffering agent sufficient to maintain the pH of the aqueous formulation between about 5 and 7.

16. A kit, comprising a formulation of any one of claims 1-15.

17. The kit of claim 16, wherein said aqueous formulation further comprises an antihistamine, decongestant, ophthalmological, antibiotic, antifungal or irrigating solution.

18. The kit of claim 16, further comprising a solid or liquid dosage form of an antihistamine, decongestant, mucolytic agent, ophthalmological, or antibiotic.

19. The kit of claim 16, further comprising a separate irrigating solution.

20. A method of treating inflammation of a nasal mucosa or paranasal mucosa in a subject, comprising intranasally administering to a subject in need thereof a therapeutically effective amount of an aqueous formulation of any of claims 1-6 or 8-15.
